(19) United States
(12) Patent Application Publication
(10) Pub. No.: US 2003/0109854 A1
(43) Pub. Date: Jun. 12, 2003

(54) Nasal cleaner

(76) Inventor: Chien-Li Chen, Taichung (TW)

Correspondence Address: BROWDY AND NEIMARK, P.L.L.C., 624 NINTH STREET, NW, SUITE 300, WASHINGTON, DC, 20001-5303, US

(21) Appl. No.: 10/011,477

(22) Filed: Dec. 11, 2001

(51) Int. Cl. A61F 5/44

(52) U.S. Cl. 604/540

(57) ABSTRACT

A nasal cleaner includes a housing mounted with a suction tip, a suction unit operated to suck air into the suction tip, and a lighting unit mounted in the housing and controlled to emit light in direction parallel to the suction tip to illuminate the nasal chamber when removing nasal mucus from the nasal chamber.

### FIELD OF THE INVENTION

The present invention relates generally to a nasal cleaner, and more particularly to a nasal cleaner, which is a combination of nasal aspirator, nasal sprayer, and light source.

### BACKGROUND OF THE INVENTION

A nasal aspirator is a device adapted to remove nasal mucus from the nasal chamber of a baby or patient who cannot remove nasal mucus by oneself. An early-developed nasal aspirator is simply a hand pump comprised of a rubber ball and a suction tip fastened to an air hole of the rubber ball. Nowadays, various motor-driven nasal aspirators have been disclosed, and have appeared on the market (for example, Taiwan Patent Utility Model #437394, issued to the present inventor). However, when examining the nasal chamber of the baby, the parent shall use a hand lamp to illuminate the nasal chamber. It is inconvenient to use a hand lamp and a nasal aspirator at the same time. Furthermore, after removal of nasal mucus, the parent may need to wash the baby's noses, the mouse, and the surrounding area. Simply using a towel or tissue paper cannot well clean the baby's skin. In order to clean the baby's skin, the towel must be wetted before washing the baby's face.

### SUMMARY OF THE INVENTION

It is one object of the present invention to provide a nasal cleaner, which provides an illumination function.

It is another object of the present invention to provide a nasal cleaner, which enables the user to remove the nasal mucus from the nasal chamber, and to clean the nasal chamber and the surrounding area with a jet flow of water.

According to one aspect of the present invention, nasal cleaner comprising a housing, the housing comprising an air chamber, the air chamber having an air inlet and an air outlet; a motor-driven suction unit mounted inside the housing and operated to suck air into the air inlet of the air chamber and force intake air out of the air outlet of the air chamber; a suction tip extended out of the housing and connected to the air inlet of the air chamber for insertion into the nasal chamber to remove nasal mucus upon operation of the motor-driven suction unit, the suction tip having an accumulation chamber adapted to collect nasal mucus; and a lighting unit, the lighting unit comprising a light hole disposed in one side of the housing adjacent to the suction tip, a light emitting element mounted in the housing and adapted to emit light through tho light hole.

According to another aspect of the present invention, the nasal cleaner as described above further comprises a water tank mounted inside the housing, a jet nozzle disposed in one side of the housing; air duct means adapted to guide air from the air chamber to the jet nozzle; water duct means adapted to guide water from the water tank to the jet nozzle; and a control knob mounted on the housing adjacent to the air outlet of the air chamber and adapted to close the air outlet of the air chamber for enabling air to pass from the air chamber through the air duct means to the jet nozzle during operation of the motor-driven suction unit.

### BRIEF DESCRIPTION OF THE DRAWINGS

FIG. 1 is a sectional view of a nasal cleaner according to a first preferred embodiment of the present invention.

FIG. 2 is a sectional view taken along line 2-2 of FIG. 1.

FIG. 3 is a sectional view of a nasal cleaner according to a second preferred embodiment of the present invention.

### DETAILED DESCRIPTION OF THE INVENTION

Referring to FIG. 1, a nasal cleaner 10 of a first preferred embodiment of the present invention is shown comprising a hollow cylindrical housing 21, a suction unit mounted inside the housing 21, and a suction tip 36.

The housing 21 comprises a frame 22 disposed on the inside near the front top side, a board 23 fastened to the frame 22, an air chamber 24 defined in between the frame 22 and the board 23. The air chamber 24 has an air inlet 25 and an air outlet 26 in the front side thereof. A valve flap 27 is provided inside the air chamber 24 for enabling air to pass through the air inlet 25 and the air outlet 26 in one direction.

The aforesaid suction unit is comprised of a diaphragm 28, a motor 31, an eccentric shaft 33, a battery 34, and a motor switch 35. The diaphragm 28 is suspended in the air chamber 24 near the rear side of the air chamber 24. The motor 32 is provided at the bottom side of the diaphragm 28, having an output shaft 32. The eccentric shaft 33 has one end coupled to the output shaft 32 of the motor 32, and the other end perpendicularly pivoted to a stem 29 at the backside of the diaphragm 28. The battery 34 is mounted near the bottom side of the housing 21, and adapted to provide the necessary working direct current power to the motor 31. The motor switch 35 is provided at the front side of the housing 21, and adapted to control the operation of the motor 31.

The suction tip 36 comprises a tubular tip body 37 fastened to the air inlet 25 and protruded over the front side of the housing 21 at a distance for insertion into the nose, and an accumulation chamber 38 disposed on the middle in fluid communication with the passage of the tubular tip body 37 and adapted to collect nasal mucus.

When the motor 31 started, the eccentric shaft 33 is driven to move the diaphragm 28 back and forth alternatively. When the diaphragm 28 moved backwards, a flow of air is sucked into the air chamber 24 through the air inlet 25. On the contrary, when the diaphragm 28 moved forwards, inside air is forced out of the air chamber 24 through the air outlet 26. By means of the reciprocating motion of the diaphragm 28, nasal mucus is sucked into the tubular tip body 37 and accumulated in the accumulation chamber 38.

The aforesaid statement is similar to a conventional nasal aspirator. The main features of the nasal cleaner 10 are described hereinafter.

The housing 21 has a light hole 41 in the front side adjacent to the suction tip 36. A light-emitting element 42 mounted in the light hole 41 and partially projecting out of the front side of the housing 21. According to the present embodiment, the light-emitting element 42 is a mini lamp bulb. A direct current (DC) power source 43 adapted to provide the necessary working DC power supply to the light-emitting element 42. According to the present embodiment, the DC power source 43 is the battery 34, i.e., the light emitting element 42 and the motor 31 use a common power source (alternatively, a separate battery may be used to provide the necessary DC power supply to the light emitting element 42, or can AC adapter may be used to convert city power supply into DC power supply for the light emitting element 42). A lamp switch 44 is provided at the front side of the housing 21 above the motor switch 35, and adapted to switch on/off the circuit between the DC power source 43 and the light-emitting element 42. A variable resistor (not shown) may be used to control the intensity of light of the light-emitting element 42.

When using the nasal cleaner 10 to remove nasal mucus from the baby, the user can switch on the lamp switch 44 to turn on the light-emitting element 42, so as to illuminate the nasal chamber for visual examination. By means of the illumination of the light-emitting element 42, the user can clean the baby's noses under a dark environment.

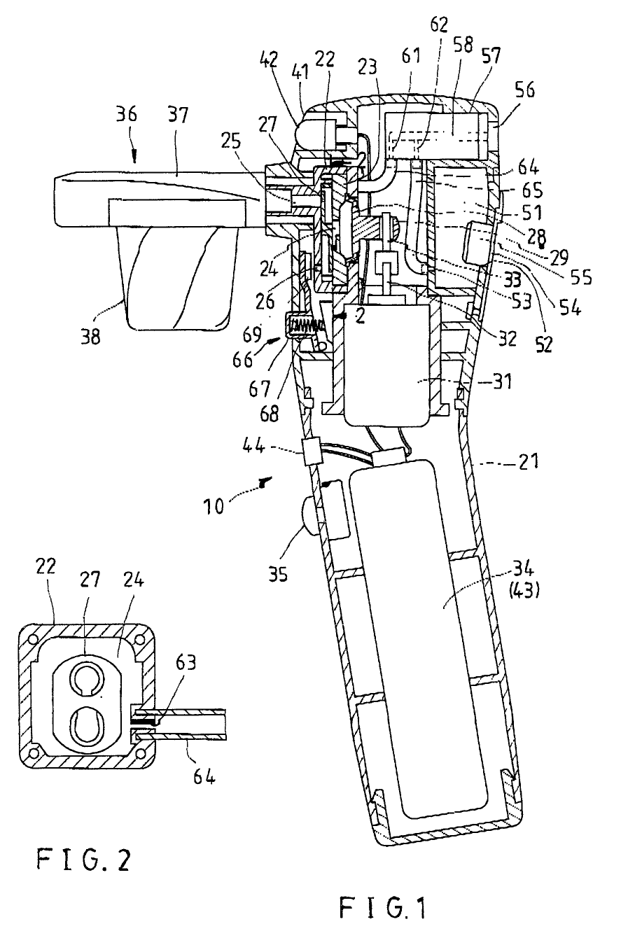

Referring to FIG. 2 and FIG. 1 again, the nasal cleaner 10 further provides a water spraying function. The housing 21 of the nasal cleaner 10 has a water tank 51 at a backside near the top. The water tank 51 has a filling hole 52 in the back sidewall thereof, and a water outlet 53 in the front sidewall thereof. A rubber strip 54 is provided having one end fixedly fastened to the housing 21 and the other end terminating in a plug 55 adapted to seal the filling hole 52. The housing 21 has a jet nozzle 56 in the back sidewall above the water tank 51. A pipe connector 57 is transversely mounted inside the housing 21 above the water tank 51. The pipe connector 57 has a main passage 58 connected to the jet nozzle 56, an air inlet 61 connected to an air outlet 63 of the air chamber 24 through an air duct (rubber tube) 64 and adapted to guide air from the air chamber 24 to the main passage 58, a water inlet 62 connected to the water outlet 53 of the water tank 51 through a water duct (rubber tube) 65 and adapted to guide water from the water tank 51 to the main passage 58. A control knob 66 is provided at the front side of the housing 21 adjacent to the air outlet 26 of the air chamber 24, and supported on a spring 68. The control knob 66 has a front operation portion 67 protruded over the front side of the housing 21, a stopping portion 69 made of a rubber flap, facing the air outlet 26.

Before the use of the nasal cleaner, clean water or saline solution is filled in the water tank 51. When the motor 31 started, the user can press the control knob 66 to force the stopping portion 69 backwards, causing the stopping portion 69 to close the air outlet 26. At this time, the intake flow of air passes through the air duct 64 and the main passage 58 of the pipe connector 57 to the jet nozzle 56 (when the air outlet 26 opened, air is directly driven out of the air outlet 26, and little amount of air passes through the long air duct 64 to the jet nozzle 56). At the same time, the rushing flow of air causes a suction force in the main passage 58 to suck water from the water tank 51 into the water duct 65 and the main passage 58, enabling intake water to be carried out of the jet nozzle 56 with the rushing flow of air.

After removal of nasal mucus from the baby's noses, use the nasal cleaner 10 to eject water through the jet nozzle 56 to clean the baby's face around the noses and the mouth. If saline solution is filled in the water tank 51, the nasal cleaner 10 can be used to clean the nasal chamber.

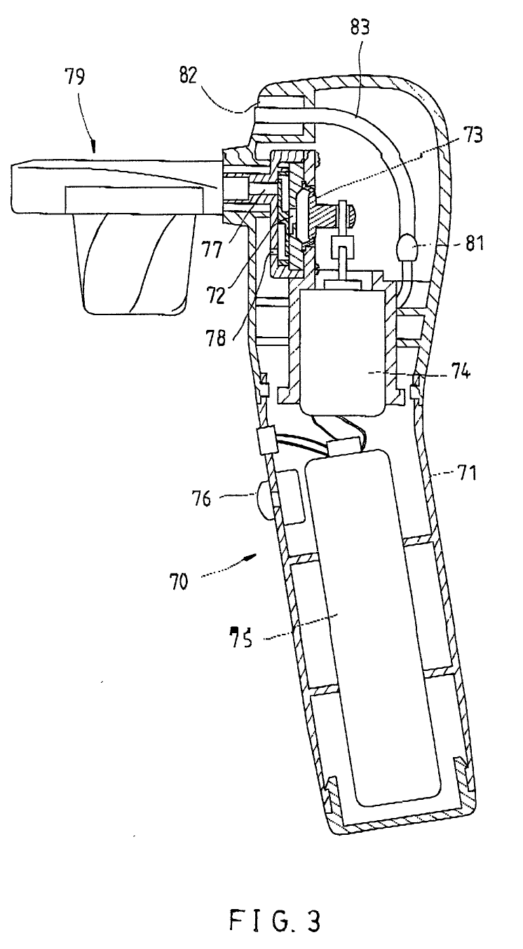

FIG. 3 shows a second embodiment of the present invention. According to this embodiment, the nasal cleaner 70 comprises a housing 71 having mounted therein an air chamber 72, a diaphragm 73, a motor 74, a battery 75, and a switch 76. When the motor 74 started, air is sucked into the suction tip 79 and the air inlet 77 and then the air chamber 72, and then driven out of the air chamber 72 through the air outlet 78. This embodiment eliminates the water spray function. According to this embodiment, a LED (light emitting diode) 81 is mounted inside the housing 71. A fiber optic 83 is connected between the LED 83 and the front light hole 82 of the housing 71 to guide light from the LED 83 to the light hole 82.

As another alternate form of the present invention, the air inlet and the light hole can be the same hole, and the fiber optic has a part extended out of the front side of the housing and disposed in parallel to the suction tip. The fiber optic can also be directly connected to the suction tip that admits light, or is made of optical fiber material.

A prototype of nasal aspirator has been constructed with the features of FIGS. 1-3. The nasal aspirator functions smoothly to provide all of the features discussed earlier.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A nasal cleaner comprising:
a housing provided with an air chamber having an air inlet and an air outlet;
a suction unit mounted inside said housing and adapted to suck air into the air inlet of said air chamber and force intake air out of said air chamber through said air outlet;
a suction tip extended out of said housing and connected to the air inlet of said air chamber for insertion into the nasal chamber of a user to remove nasal mucus upon operation of said suction unit, said suction tip having an accumulation chamber adapted to collect nasal mucus;
wherein the nasal cleaner further comprises a light hole disposed in one side of said housing, a light emitting element mounted in said housing and adapted to emit light through said light hole, and a lamp switch mounted in said housing and partially projecting out of said housing and adapted to control the operation of said light emitting element.

2. The nasal cleaner as claimed in claim 1 wherein said light-emitting element is installed in said light hole.

3. The nasal cleaner as claimed in claim 1 wherein said cleaner further comprises a fiber optic connected between said light emitting element and said light hole and adapted to guide light from said light emitting element to said light hole.

4. The nasal cleaner as claimed in claim 1 wherein said lamp switch controls the intensity of light of said light emitting element.

5. The nasal cleaner as claimed in claim 1 wherein said cleaner further comprises a power source, which provides the necessary power supply to said light emitting element.

6. The nasal cleaner as claimed in claim 1 further comprising:
a water tank mounted inside said housing, said water tank having a water-filling hole;
seal means adapted to close the water-filling hole of said water tank;
a jet nozzle disposed in one side of said housing;
an air duct communicated between said air chamber and said jet nozzle;
a water duct communicated between said water tank and said jet nozzle; and
a control knob mounted on said housing adjacent to the air outlet of said air chamber, said control knob having a front operation portion protruded to the outside of said housing and a stopping portion adapted to close the air outlet of said air chamber when the user depresses said front operation portion.

7. The nasal cleaner as claimed in claim 6 wherein said seal means comprises a flexible strip fasten on said housing adjacent to said water filling hole, and a plug integral with said rubber strip and adapted to close said water filling hole.

8. The nasal cleaner as claimed in claim 6 further comprising a pipe connector fixedly mounted inside said housing, said pipe connector having a main passage connected to said jet nozzle, a water inlet in communication with said main passage, and an air inlet in communication with said main passage, said water duct is connected to said water inlet of said pipe connector and adapted to guide water from said water tank to said jet nozzle, and said air duct is connected to said air inlet of said pipe connector and adapted to guide air from said air chamber to said jet nozzle.

9. The nasal cleaner as claimed in claim 6 wherein said control knob is supported on a spring inside said housing, said control knob having a front side protruded to the outside of said housing to form said operation portion and a rubber flap disposed at the opposite of said operation portion to form said stopping portion.
